SEQUENCE LISTING (8 sequences)

SEQ ID NO: 1 - protein, 431 aa, Artificial Sequence
  Protein
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 432 aa, Artificial Sequence
  Protein
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 339 aa, Artificial Sequence
  Protein
LTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 340 aa, Artificial Sequence
  Protein
LTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 92 aa, Artificial Sequence
  Protein
SDTGRPFVEMYSEIPEIIHMTEGRELVIPCRVTSPNITVTLKKFPLDTLIPDGKRIIWDSRKGFIISNATYKEIGLLTCEATVNGHLYKTNY

SEQ ID NO: 6 - protein, 278 aa, Artificial Sequence
  Protein
DLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 279 aa, Artificial Sequence
  Protein
DLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 153 aa, Artificial Sequence
  Protein
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNR

(19) United States
(12) Patent Application Publication
(10) Pub. No.: US 2022/0098279 A1
(43) Pub. Date: Mar. 31, 2022

(54) AFLIBERCEPT ATTRIBUTES AND METHODS OF CHARACTERIZING AND MODIFYING THEREOF

(71) Applicant: Amgen Inc., Thousand Oaks, CA (US)

(72) Inventors: Quanzhou LUO, Oak Park, CA (US); Scott KUHNS, Newbury Park, CA (US); Diana WOEHLE, Simi Valley, CA (US); Matthew JERUMS, Oak View, CA (US); XueJun HAN, Thousand Oaks, CA (US); Kelli M.G. MATTHIES, Newbury Park, CA (US)

(21) Appl. No.: 17/426,886

(22) PCT Filed: Jan. 29, 2020
(86) PCT No.: PCT/US2020/015659

§ 371 Date: Jul. 29, 2021

Related U.S. Application Data
(60) Provisional application No. 62/798,903, filed on Jan. 30, 2019.

(51) Int. Cl. C07K 14/71; C07K 1/16

(52) U.S. Cl. CPC C07K 14/71; C07K 2319/30; C07K 2317/41; C07K 1/165

(57) ABSTRACT

The present disclosure relates to aflibercept, in particular, attributes of aflibercept. Also provided herein are methods of characterizing and modifying the attributes of aflibercept and compositions comprising aflibercept with particular attributes.

### RELATED APPLICATIONS

This application claims the benefit of U.S. Provisional Application No. 62/798,903, filed on Jan. 30, 2019, which is hereby incorporated by reference in its entirety.

### SEQUENCE LISTING

The present application is being filed along with a Sequence Listing in electronic format. The Sequence Listing is provided as a file entitled A-2287-WO-PCT_SeqList.txt, created Jan. 29, 2020, which is 20.5 kb in size. The information in the electronic format of the Sequence Listing is incorporated herein by reference in its entirety.

### FIELD OF THE INVENTION

The instant disclosure relates to aflibercept, in particular, attributes of aflibercept. Also provided herein are methods of characterizing and modifying the attributes of aflibercept and compositions comprising aflibercept with particular attributes.

### BACKGROUND

Vascular endothelial growth factor (VEGF), also referred to as VEGF-A, is a signaling protein that promotes the growth of new blood vessels and binds to VEGFR-1 and VEGFR-2. VEGF has been shown to be upregulated in many tumors and has a role in angiogenesis. VEGF has also been shown to have a role in intraocular neovascularization, such as choroidal neovascularization (CNV), which is a significant aspect of wet age-related macular degeneration (AMD).

VEGF inhibitors, such as anti-VEGF antibodies and fragments and decoy receptors or chimeric receptors, have been developed as therapeutics for the treatment of various conditions, such as cancer and ocular disorders. For example, an anti-VEGF antibody and an anti-VEGF Fab are both commercially available as bevacizumab and ranibizumab, respectively. Also, commercially available is aflibercept, a VEGFR-Fc fusion protein or “VEGF-trap.”

Aflibercept is a fusion protein composed of an IgG1 Fc domain fused to the Ig domain 2 of VEGFR-1 and Ig domain 3 of VEGFR-2. Aflibercept is marketed as Eylea® (Regeneron, Tarrytown, N.Y.) for the treatment of various ocular conditions, including wet type AMD, and is formulated for intravitreal administration. The fusion protein is also marketed as Zaltrap® (ziv-aflibercept) (Regeneron, Tarrytown, N.Y.) for the treatment of certain types of cancer and is formulated for intravenous administration.

An attribute of a protein can have an important role in at least the quality of a protein product. Accordingly, provided herein are methods of characterizing and modifying the attributes of aflibercept and compositions comprising aflibercept with attributes, and related advantages.

### SUMMARY

The present disclosure provides compositions of aflibercept, including compositions comprising a mixture of aflibercept species. Also provided herein are aflibercept species. In one embodiment, a species of aflibercept has one or more different attributes than another species of aflibercept. In some embodiments, the difference is the presence or absence of an attribute. In another embodiment, the difference is the level or amount of an attribute. Also, provided herein are methods of characterizing one or more attributes of aflibercept as well as methods of modifying one or more attributes of aflibercept, purifying aflibercept, and producing compositions of aflibercept.

One aspect of the present disclosure is an aflibercept species that is a Y92L clipped species of aflibercept. The Y92L clipped species can comprise SEQ ID NO: 3.

Another aspect of the present disclosure is a composition comprising a mixture of aflibercept species, including the Y92L clipped species. In one embodiment, the amount of Y92L clipped species in the composition is less than 5.0%, as determined by reduced capillary electrophoresis sodium dodecyl sulfate (rCE-SDS). In one embodiment, the amount of Y92L clipped species in the composition is less than 0.8%, as determined by rCE-SDS. In some embodiments, the amount of Y92L clipped species in the composition is less than 3.0%, between 1.0% and 5.0% or between 1.0% and 3.0%, about 1.1%, about 3.0% or about 4.7% of the composition, as determined by rCE-SDS. In one embodiment, the amount of Y92L clipped species in the composition is about 0.4%, as determined by rCE-SDS.

In another aspect of the present disclosure, the composition comprising a mixture of aflibercept species comprises N68 occupied species of aflibercept. In one embodiment, at least 30% of the aflibercept species is occupied at position N68, as determined by rCE-SDS. In some embodiments, at least 50%, between 50-60%, about 39%, about 53%, about 54%, or about 55% of the aflibercept species is occupied at position N68. In some embodiments, the composition also comprises Y92L clipped species, such as between 1% and 3%, less than 0.8%, about 1.1% or about 0.4% of the composition.

The present disclosure also provides a composition comprising a mixture of aflibercept species, wherein the total sialic acid content of aflibercept species is between 6.0 and 10.0 mol/mol protein, as determined by LC-MS based peptide mapping. In one embodiment, the total sialic acid content is about 6.8, about 8.5 or about 9.5 mol/mol protein. In some embodiments, the total sialic acid content is about 9.5 mol/mol protein, and wherein between 54-55% of the aflibercept species is occupied at position N68, and wherein the amount of Y92L clipped species in the mixture is about 1.1%.

Another aspect of the present disclosure is a composition comprising a mixture of aflibercept species, wherein the aflibercept species comprise less than 13% afucosylation in the Fc domain. The percentage of afucosylation can be determined by any method known in the art, such as LC-MS based peptide mapping. In one embodiment, the aflibercept species comprise between 1.0-12% afucosylation in the Fc domain. In another embodiment, the aflibercept species comprise less than 12%, 11%, 10%, 9%, 8%, 7%, 6%, or 5% afucosylation in the Fc domain. In one embodiment, the aflibercept species comprise less than 10% afucosylation in the Fc domain. In another embodiment, the aflibercept species comprise less than 5% afucosylation in the Fc domain. In another embodiment, the aflibercept species comprise about 12%, 11%, 10%, 9%, 8%, 7%, 6%, 5% or 4% afucosylation in the Fc domain. In yet another embodiment, the aflibercept species comprise about 4% afucosylation in the Fc domain.

Another aspect of the present disclosure is a composition comprising a mixture of aflibercept species, wherein the aflibercept species comprise between 0.1-2.0 (mol/mol protein) of sialic acid at N68; between 1.0-25.0% O-glycosylation at T33; between 70%-99% N-glycosylation at N36; between 20%-75% of N-glycosylation at N68; between 0.1-1.0% high mannose in the Fc domain; between 26% galactosylation in the Fc domain; about 1% sialylation in the Fc domain; or any combination thereof. The aflibercept species can further comprise less than 13% afucosylation in the Fc domain, such as between 1.0-12% afucosylation in the Fc domain, or about 12%, 11%, 10%, 9%, 8%, 7%, 6%, 5% or 4% afucosylation in the Fc domain, such as less than 5% afucosylation in the Fc domain. In one embodiment, the aflibercept species comprise about 4% afucosylation in the Fc domain.

The aflibercept species in a mixture can also comprise about 0.7 (mol/mol protein) of sialic acid at N68; about 4% O-glycosylation at T33; about 95% N-glycosylation at N36; about 26% of N-glycosylation at N68; about 1% high mannose in the Fc domain; about 26% galactosylation in the Fc domain; about 1% sialylation in the Fc domain; less than 13% afucosylation in the Fc domain, such as between 1.0-12% afucosylation in the Fc domain, or about 12%, 11%, 10%, 9%, 8%, 7%, 6%, 5% or 4% afucosylation in the Fc domain; or any combination thereof. In one embodiment, the aflibercept species in a mixture comprises about 0.7 (mol/mol protein) of sialic acid at N68; about 4% O-glycosylation at T33; about 95% N-glycosylation at N36; about 26% of N-glycosylation at N68; about 1% high mannose in the Fc domain; about 6% afucosylation in the Fc domain; about 26% galactosylation in the Fc domain; about 1% sialylation in the Fc domain; or any combination thereof.

The aflibercept species in a mixture can also comprise about 0.7 (mol/mol protein) of sialic acid at N68; about 4% O-glycosylation at T33; about 95% N-glycosylation at N36; about 26% of N-glycosylation at N68; about 1% high mannose in the Fc domain; about 26% galactosylation in the Fc domain; about 1% sialylation in the Fc domain; less than 13% afucosylation in the Fc domain, such as between 1.0-12% afucosylation in the Fc domain, or about 12%, 11%, 10%, 9%, 8%, 7%, 6%, 5% or 4% afucosylation in the Fc domain; less than 0.8% of Y92L clipped species, such as about 0.4%; or any combination thereof.

The aflibercept species in a mixture can also comprise about 0.8 (mol/mol protein) of sialic acid at N68; about 8.3% O-glycosylation at T33; about 90.9% N-glycosylation at N36; about 51.9% of N-glycosylation at N68; about 0.4% high mannose in the Fc domain; about 24.4% galactosylation in the Fc domain; about 3.8% sialylation in the Fc domain; less than 13% afucosylation in the Fc domain, such as between 1.0-12% afucosylation in the Fc domain, or about 12%, 11%, 10%, 9%, 8%, 7%, 6%, 5% or 4% afucosylation in the Fc domain; or any combination thereof. In one embodiment, the aflibercept species in a mixture comprises about 0.8 (mol/mol protein) of sialic acid at N68; about 8.3% O-glycosylation at T33; about 90.9% N-glycosylation at N36; about 51.9% of N-glycosylation at N68; about 0.4% high mannose in the Fc domain; about 4% afucosylation in the Fc domain; about 24.4% galactosylation in the Fc domain; about 3.8% sialylation in the Fc domain; or any combination thereof.

The aflibercept species in a mixture can also comprise about 0.8 (mol/mol protein) of sialic acid at N68; about 8.3% O-glycosylation at T33; about 90.9% N-glycosylation at N36; about 51.9% of N-glycosylation at N68; about 0.4% high mannose in the Fc domain; about 24.4% galactosylation in the Fc domain; about 3.8% sialylation in the Fc domain; less than 13% afucosylation in the Fc domain, such as between 1.0-12% afucosylation in the Fc domain, or about 12%, 11%, 10%, 9%, 8%, 7%, 6%, 5% or 4% afucosylation in the Fc domain; less than 0.8% of Y92L clipped species, such as about 0.4%; or any combination thereof. In one embodiment, the aflibercept species in a mixture comprises about 0.8 (mol/mol protein) of sialic acid at N68; about 8.3% O-glycosylation at T33; about 90.9% N-glycosylation at N36; about 51.9% of N-glycosylation at N68; about 0.4% high mannose in the Fc domain; about 4% afucosylation in the Fc domain; about 24.4% galactosylation in the Fc domain; about 3.8% sialylation in the Fc domain; about 0.4% of Y92L clipped species; or any combination thereof. In one embodiment, the aflibercept species in a mixture comprises about 0.8 (mol/mol protein) of sialic acid at N68; about 8.3% 0-glycosylation at T33; about 90.9% N-glycosylation at N36; about 51.9% of N-glycosylation at N68; about 0.4% high mannose in the Fc domain; about 4% afucosylation in the Fc domain; about 24.4% galactosylation in the Fc domain; about 3.8% sialylation in the Fc domain; and about 0.4% of Y92L clipped species.

The present disclosure also provides a method of increasing the binding of a composition comprising a mixture of aflibercept species to placental growth factor (PlGF) and/or VEGF-A comprising reducing the amount of Y92L clipped species in the mixture or by reducing the N68 occupancy of aflibercept. The PlGF can be PlGF-1 or PlGF-2. In one embodiment, the method of reducing the amount of Y92L clipped species in the mixture comprises purifying aflibercept with an anion exchange chromatography step followed by a hydrophobic interaction chromatography step.

Another aspect of the present disclosure is a method of producing a composition comprising a mixture of aflibercept species. The method can comprise subjecting a cell culture fluid comprising a mixture of aflibercept species to a purification process comprising an anion exchange chromatography (AEX) step followed by a hydrophobic interaction chromatography (HIC) step, wherein a lower amount of Y92L clipped species is produced as compared to a purification process comprising an AEX step followed by a cation exchange chromatography (CEX) step. In some embodiments, the purification process does not include a CEX step.

Another method of producing a composition comprising a mixture of aflibercept species comprises harvesting cells producing aflibercept, subjecting the harvested cell culture fluid to a Protein A column, and performing anion exchange chromatography (AEX) followed by cation exchange chromatography (CEX) or hydrophobic interaction chromatography (HIC). In some embodiments, AEX is followed by HIC, and optionally, the method does not include CEX. In some embodiments, AEX is followed by CEX. In one embodiment, the cells are Chinese Hamster Ovary (CHO) cells, which can be grown by perfusion or fed-batch. The cells can be harvested by acid precipitation, centrifugation, microfiltration, depth filtration or any combination thereof. The method can also further comprise one or more viral inactivation and/or viral filtration step(s), or ultrafiltration/diafiltration (UF/DF) step. In some embodiments, the method does not comprise performing CEX prior to performing AEX, HIC, and/or size exclusion chromatography (SEC).

### BRIEF DESCRIPTION OF THE DRAWINGS

FIG. 1 depicts attributes of aflibercept mapped onto its structure.

FIG. 2 is a graph of the relative VEGF-A binding (%) as a function of the percentage of Y92L clipped species of aflibercept.

FIG. 3 is a graph of the relative PlGF-1 binding (%) as a function of the percentage of N68 occupancy of aflibercept.

FIG. 4 depicts 2 versions of a process to produce aflibercept. In the first process, AEX chromatography is followed by CEX. In the second process, AEX chromatography is followed by HIC.

FIG. 5 depicts the reduced capillary electrophoresis-sodium dodecyl sulfate (rCE-SDS) results of aflibercept from hydrophobic interaction chromatography (HIC) fractions (F1-F5), along with the aflibercept species represented by each peak.

FIG. 6 depicts the reduced capillary electrophoresis-sodium dodecyl sulfate (rCE-SDS) results of aflibercept manufactured with a process using anion exchange followed by cation exchange (DS) and aflibercept manufactured with a process using anion exchange followed by hydrophobic interaction chromatography.

FIG. 7 depicts the percentage of Y92L clipped species of aflibercept grown in various culture conditions.

### DETAILED DESCRIPTION

The instant disclosure provides a composition comprising a mixture of aflibercept species as well as various species of aflibercept. In some embodiments, a species of aflibercept has one or more different attributes than another species of aflibercept. Also, provided herein are methods of characterizing one or more attributes of aflibercept as well as modifying one or more attributes of aflibercept.

In one embodiment, aflibercept comprises an amino acid sequence of SEQ ID NO: 1, in which the C-terminal lysine is not present. In another embodiment, aflibercept comprises an amino acid sequence of SEQ ID NO: 2, in which the C-terminal lysine is present.

SEQ ID NO: 1
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG
SEQ ID NO: 2
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK

Also provided herein is one or more attributes of aflibercept. The attribute can comprise deamidation, glycosylation (e.g., O-glycosylation, N-glycosylation, sialylation (e.g., NANA or NGNA sialylation), afucosylation, mannosylation (e.g., high mannose), galactosylation, or clipping (e.g., at the C-terminal or the N-terminus). In one embodiment, an attribute of aflibercept is characterized by the level or amount of deamidation, glycosylation (e.g., O-glycosylation, N-glycosylation, sialylation (e.g., NANA or NGNA sialylation), afucosylation, mannosylation (e.g., high mannose), galactosylation, clipping (e.g., at the C-terminal or the N-terminus), or any combination thereof, of a composition comprising a mixture of aflibercept species.

In some embodiments, the characterization of an attribute of aflibercept is by the level or amount of deamidation, glycosylation (e.g., O-glycosylation, N-glycosylation, sialylation (e.g., NANA or NGNA sialylation), afucosylation, mannosylation (e.g., high mannose), galactosylation, clipping (e.g., at the C-terminal or the N-terminus), or any combination thereof, of the VEGFR domains of a composition comprising a mixture of aflibercept species. In another embodiment, the characterization of an attribute of aflibercept is by the level or amount of deamidation, glycosylation (e.g., O-glycosylation, N-glycosylation, sialylation (e.g., NANA or NGNA sialylation), afucosylation, mannosylation (e.g., high mannose), galactosylation, clipping (e.g., at the C-terminal or the N-terminus), or any combination thereof, of the Fc domains of a composition comprising a mixture of aflibercept species. In one embodiment, the attributes are the level or amount of sialylation and/or glycosylation of the VEGFR domains. In one embodiment, the attributes are the level or amount of sialylation, N-glycosylation, O-glycosylation, or any combination thereof, of the VEGFR domains. In another embodiment, the attributes are the level or amount of high mannose, sialylation, afucosylation, galactosylation or any combination thereof, of the Fc domain.

In another embodiment, characterization of an attribute of aflibercept is by the percentage of deamidation, glycosylation (e.g., O-glycosylation, N-glycosylation, sialylation (e.g., NANA or NGNA sialylation), afucosylation, mannosylation (e.g., high mannose), galactosylation, clipping (e.g., at the C-terminal or the N-terminus), or any combination thereof, of a composition comprising a mixture of aflibercept species. In another embodiment, characterization of an attribute of aflibercept is by the number of moles of a glycan (e.g., sialic acid) per mole of aflibercept or aflibercept species of a composition comprising a mixture of aflibercept species.

In some embodiments, the attribute is at a specific amino acid position of aflibercept. For example, the attribute can be the level or amount of deamidation at N84 and/or N99; sialic acid at N36, N88, N123, N196, or any combination thereof; 0-glycosylation at T33; N-glycosylation at N36, N68, N123, N196, or any combination thereof; clipping at Y92L (e.g., resulting in an aflibercept species having an amino acid sequence of SEQ ID NO: 3 or SEQ ID NO: 4, which lacks the initial 92 amino acid (SEQ ID ON: 5) of SEQ ID NO: 1); clipping at R153D (e.g., resulting in an aflibercept species having an amino acid sequence of SEQ ID NO: 6 or SEQ ID NO: 7, which lacks the initial 153 amino acids (SEQ ID ON: 8) of SEQ ID NO: 1); or any combination thereof. In some embodiments, clipping at R153D results in a protein comprising an amino acid sequence of SEQ ID NO: 1, in which the initial 153 amino acids (SEQ ID NO: 8) that is clipped, is joined to the protein through a disulfide bond.

SEQ ID NO: 3
LTHRQTNTIIDVVLSPSHGIELSVGEKLVLNCTARTELNVGIDFNWEYPS
SKHQHKKLVNRDLKTQSGSEMKKFLSTLTIDGVTRSDQGLYTCAASSGLM
TKKNSTFVRVHEKDKTHTCPPCPAPELLGGPSVFLFPPKPKDTLMISRTP
EVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLY
SKLTVDKSRWQQGNVFSCSVMHEALHNHYTQKSLSLSPG
SEQ ID NO: 4
LTHRQTNTIIDVVLSPSHGIELSVGEKLVLNCTARTELNVGIDFNWEYPS
SKHQHKKLVNRDLKTQSGSEMKKFLSTLTIDGVTRSDQGLYTCAASSGLM
TKKNSTFVRVHEKDKTHTCPPCPAPELLGGPSVFLFPPKPKDTLMISRTP
EVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 5 (amino acids 1-92 of SEQ ID NO: 1)
SDTGRPFVEMYSEIPEIIHMTEGRELVIPCRVTSPNITVTLKKFPLDTLI
PDGKRIIWDSRKGFIISNATYKEIGLLTCEATVNGHLYKTNY
SEQ ID NO: 6
DLKTQSGSEMKKFLSTLTIDGVTRSDQGLYTCAASSGLMTKKNSTFVRVH
EKDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQ
QGNVFSCSVMHEALHNHYTQKSLSLSPG
SEQ ID NO: 7
DLKTQSGSEMKKFLSTLTIDGVTRSDQGLYTCAASSGLMTKKNSTFVRVH
EKDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQ
QGNVFSCSVMHEALHNHYTQKSLSLSPGK
SEQ ID NO: 8 (amino acids 1-153 of SEQ ID NO: 1)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNR

Accordingly, also provided herein is aflibercept with one or more of an attribute disclosed herein. In some embodiments, a species of aflibercept has one or more attributes that differs from another species of aflibercept. The difference can be the presence or absence of an attribute or the level or amount of an attribute. In one embodiment, an aflibercept species is deamidated at N84 and/or N99; sialylated at N36, N88, N123, N196, or any combination thereof 0-glycosylated at T33; N-glycosylated at N36, N68, N123, N196, or any combination thereof or any combination thereof.

In another embodiment, an aflibercept species is clipped at Y92L (e.g., resulting in an aflibercept species having an amino acid sequence of SEQ ID NO: 3 or SEQ ID NO: 4, which lacks the initial 92 amino acid (SEQ ID ON: 5) of SEQ ID NO: 1). In some embodiments, the clipped Y92L species is deamidated at N99; sialylated at N123 and/or N196; N-glycosylated at N123 and/or N196, or any combination thereof.

In another embodiment, an aflibercept species is clipped at R153D (e.g., resulting in an aflibercept species having an amino acid sequence of SEQ ID NO: 6 or SEQ ID NO: 7, which lacks the initial 153 amino acids (SEQ ID ON: 8) of SEQ ID NO: 1). In some embodiments, the clipped R153D species is sialylated at N196 and/or N-glycosylated at N196.

Also provided herein are compositions comprising a mixture of aflibercept species with particular attributes. In some embodiments, less than 10%, 9%, 8%, 7%, 6%, 5%, 4%, 3%, 2%, 1%, or 0.8% of the aflibercept species is of a clipped species (e.g., Y92L or R153D clipped species). The amount of clipped species can be determined by any method known in the art, such as rCE-SDS or trypsin peptide mapping. In one embodiment, the composition comprises a mixture of aflibercept species in which less than 5.0% of the aflibercept species is a Y92L clipped species In some embodiments, less than 10%, 9%, 8%, 7%, 6%, 5%, 4%, 3%, 2%, 1% or 0.8% of the aflibercept species is a Y92L clipped species. In one embodiment, the composition comprises a mixture of aflibercept species in which less than 0.8% of the aflibercept species is a Y92L clipped species. In another embodiment, the composition comprises a mixture of aflibercept species in which less than 0.5% of the aflibercept species is a Y92L clipped species. In some embodiments, the amount of Y92L clipped species in the composition is between 1% and 10%, between 1.0% and 5.0% or between 1.0% and 3.0%. In some embodiments, the amount of Y92L clipped species in the composition is about 1.0%, about 2.0%, about 3.0%, about 4.0%, about 5.0%, or about 6.0% of the composition. In some embodiments, the amount of Y92L clipped species in the composition is about 1.1%, about 3.0% or about 4.7% of the composition. In one embodiment, the composition comprises a mixture of aflibercept species in which about 0.4% of the aflibercept species is a Y92L clipped species.

Also provided herein are compositions comprising a mixture of aflibercept species wherein at least 10%, 20%, 30%, 40%, 50%, 60%, 70%, 80%, or 90% of the aflibercept species is occupied (i.e., glycosylated) at position N68. The amount of aflibercept species occupied at position N68 can be determined by any method known in the art, such as rCE-SDS or peptide mapping. In some embodiments, between 10% and 90%, between 20% and 70%, between 30% and 60% or between 50-60% of the aflibercept species is occupied at position N68. In some embodiments, the amount of aflibercept species occupied at position N68 is about 39%, about 53%, about 54%, or about 55%.

Also provided herein are compositions comprising a mixture of aflibercept species wherein the total sialic acid content of aflibercept species is between 1.0 and 20.0 mol/mol protein, between 2.0 and 15.0 mol/mol protein, between 5.0 and 12.0 mol/mol protein, or between 6.0 and 10.0 mol/mol protein. The amount of sialic acid can be determined by any method known in the art, such as any peptide mapping or glycan analysis method, such as liquid chromatography coupled with mass spectrometry (LC-MS) based peptide mapping, total sialic acid method, or total hydrophilic-interaction liquid chromatography (HILIC) glycan mapping. In one embodiment, the total sialic acid content is about 6.8, about 8.5 or about 9.5 mol/mol protein.

In another embodiment, the composition comprises a mixture of aflibercept species in which the glycan profile of the mixture comprises about 0.7 sialic acid (mol/mol protein) at N88, about 4% O-glycosylation at T33, about 95% N-glycosylation at N36, about 26% N-glycosylation at N68, about 1% high mannose at its Fc domain, about 6% afucosylation at its Fc domain, about 26% galactosylation of its Fc domain, about 1% sialylation at its Fc domain, or any combination thereof. In some embodiments, the glycan profile can further comprise about 3.2 sialic acid (mol/mol protein) at N36, about 1.6 sialic acid (mol/mol protein) at N123, about 1.9 sialic acid (mol/mol protein) at N196, total sialic acid (mol/mol protein) of about 7.4 at its VEGFR domains, about 100% N-glycosylation at N123, about 99% N-glycosylation at N196, or any combination thereof.

In one embodiment, the composition comprises a mixture of aflibercept species in which the glycan profile of the mixture comprises about 3.2 sialic acid (mol/mol protein) at N36, about 0.7 sialic acid (mol/mol protein) at N88, about 1.6 sialic acid (mol/mol protein) at N123, about 1.9 sialic acid (mol/mol protein) at N196, total sialic acid (mol/mol protein) of about 7.4 at its VEGFR domains, about 4% O-glycosylation at T33, about 95% N-glycosylation at N36, about 26% N-glycosylation at N68, about 100% N-glycosylation at N123, about 99% N-glycosylation at N196, about 1% high mannose at its Fc domain, about 6% afucosylation at its Fc domain, about 26% galactosylation of its Fc domain, about 1% sialylation at its Fc domain, or any combination thereof.

In some embodiments, the composition comprises a mixture of aflibercept species in which the amount of Y92L clipped species in the composition is less than 10%, 9%, 8%, 7%, 6%, 5%, 4%, 3%, 2%, 1%, 0.8%; between 1% and 10%, between 1.0% and 5.0% or between 1.0% and 3.0%; about 1.0%, about 2.0%, about 3.0%, about 4.0%, about 5.0%, or about 6.0%; or about 1.1%, about 3.0%, about 4.7%, or about 0.4%; and the glycan profile of the mixture of aflibercept comprises about 0.7 sialic acid (mol/mol protein) at N88, about 4% O-glycosylation at T33, about 95% N-glycosylation at N36, about 26% N-glycosylation at N68, about 1% high mannose at its Fc domain, about 6% afucosylation at its Fc domain, about 26% galactosylation of its Fc domain, about 1% sialylation at its Fc domain, or any combination thereof. In some embodiments, the glycan profile further comprises about 3.2 sialic acid (mol/mol protein) at N36, about 1.6 sialic acid (mol/mol protein) at N123, about 1.9 sialic acid (mol/mol protein) at N196, total sialic acid (mol/mol protein) of about 7.4 at its VEGFR domains, about 100% N-glycosylation at N123, about 99% N-glycosylation at N196, or any combination thereof. In some embodiments, the composition further comprises at least 10%, 20%, 30%, 40%, 50%, 60%, 70%, 80%, or 90%; between 10% and 90%, between 20% and 70%, between 30% and 60% or between 50-60%; or about 39%, about 53%, about 54%, or about 55%; of the aflibercept species is occupied (i.e., glycosylated) at position N68. In some embodiments, the composition further comprises a total sialic acid content of aflibercept species that is between 1.0 and 20.0 mol/mol protein, between 2.0 and 15.0 mol/mol protein, between 5.0 and 12.0 mol/mol protein, between 6.0 and 10.0 mol/mol protein, about 6.8, about 8.5 or about 9.5 mol/mol protein.

In one embodiment, the composition comprises a mixture of aflibercept species in which at least 10%, 20%, 30%, 40%, 50%, 60%, 70%, 80%, or 90%; between 10% and 90%, between 20% and 70%, between 30% and 60% or between 50-60%; or about 39%, about 53%, about 54%, or about 55%; of the aflibercept species is occupied (i.e., glycosylated) at position N68; and the glycan profile of the aflibercept species in the composition comprises about 0.7 sialic acid (mol/mol protein) at N88, about 4% 0-glycosylation at T33, about 95% N-glycosylation at N36, about 26% N-glycosylation at N68, about 1% high mannose at its Fc domain, about 6% afucosylation at its Fc domain, about 26% galactosylation of its Fc domain, about 1% sialylation at its Fc domain, or any combination thereof. In some embodiments, the glycan profile further comprises about 3.2 sialic acid (mol/mol protein) at N36, about 1.6 sialic acid (mol/mol protein) at N123, about 1.9 sialic acid (mol/mol protein) at N196, total sialic acid (mol/mol protein) of about 7.4 at its VEGFR domains, about 100% N-glycosylation at N123, about 99% N-glycosylation at N196, or any combination thereof. In some embodiments, the composition further comprises a total sialic acid content of aflibercept species that is between 1.0 and 20.0 mol/mol protein, between 2.0 and 15.0 mol/mol protein, between 5.0 and 12.0 mol/mol protein, between 6.0 and 10.0 mol/mol protein, about 6.8, about 8.5 or about 9.5 mol/mol protein.

In one embodiment, the composition comprises a mixture of aflibercept species in which the amount of Y92L clipped species in the mixture is between 1% and 3% (e.g., about 1.1%, less than 0.8%, or about 0.4%), between 53-55% (e.g., about 54-55%) of the aflibercept species is occupied at position N68, and the total sialic acid content is about 9.5 mol/mol protein.

Also provided herein are methods of producing a composition comprising a mixture of aflibercept species. Aflibercept can be produced by a host cell, such as a mammalian host cell. The mammalian host cell can be a Chinese Hamster Ovary (CHO) cell. In one embodiment, the method comprises culturing and harvesting host cells producing aflibercept and purifying the protein with Protein A followed by anion exchange chromatography (AEX) and then cation exchange chromatography (CEX). In another embodiment, the method comprises culturing the host cells by fed batch, harvesting the cells, and purifying the protein with Protein A followed by anion exchange chromatography (AEX) and then hydrophobic interaction chromatography (HIC). In some embodiments, the method comprising AEX followed by HIC produces a lower amount of Y92L clipped species as compared to the same method but with CEX instead of HIC. In some embodiments, the method comprising AEX followed by HIC produces more than two-fold less Y92L clipped species than the same method but with CEX instead of HIC. In some embodiments, the method comprising AEX followed by HIC does not include a CEX step. In some embodiments, the method does not comprise any size exclusion chromatography (SEC) steps. In some embodiments, the method does not comprise any CEX step before AEX.

In some embodiments, the method comprises culturing the host cells by fed-batch or perfusion. In some embodiments, such as when culturing by a method comprising perfusion, the cells can be harvested by flocculation, microfiltration, or any combination thereof. In some embodiments, such as when culturing by fed-batch, the cells can be harvested by precipitation, centrifugation, depth filtration or any combination thereof. In one embodiment, the cells are harvested by acid precipitation. The acid can be any known in the art, such as acetic acid or citric acid. In one embodiment, acid precipitation is at a pH of approximately 5.5, approximately 5.0, approximately 4.5, approximately 4.0, approximately 3.5, or approximately 3.0. In one embodiment, acid precipitation is at a pH of approximately 4.1, approximately 4.2, approximately 4.3, approximately 4.4, approximately 4.5, approximately 4.6, approximately 4.7, approximately 4.8, approximately 4.9, or approximately 5.0. In one embodiment, acid precipitation is at a pH of 3.0-4.0 or 3.5-4.0. In another embodiment, the pH is 5.5±0.2, 5.4±0.2, 5.3±0.2, 5.2±0.2, 5.1±0.2, 5.0±0.2, 4.9±0.2, 4.8±0.2, 4.7±0.2, 4.6±0.2, 4.5±0.2, 4.4±0.2, 4.3±0.2, 4.2±0.2, 4.1±0.2, 4.0±0.2, 4.0±0.2, 3.9±0.2, 3.8±0.2, 3.7±0.2, 3.6±0.2, 3.5±0.2, 3.4±0.2, 3.3±0.2, 3.2±0.2, 3.1±0.2, or 3.0±0.2. Acid precipitation can be at a temperature of about 20° C., 15° C., 10° C., or 5° C. In one embodiment, the temperature is about 20±3° C., 19±3° C., 18±3° C., 17±3° C., 16±3° C., 15±3° C., 14±3° C., 13±3° C., 12±3° C., 11±3° C., 10±3° C., 9±3° C., 8±3° C., 7±3° C., 6±3° C., 5±3° C., or 4±3° C. The acid precipitation process can be for about 30 minutes, 45 minutes, 60 minutes, 75 minutes, 90 minutes, or 105 minutes.

In another embodiment, the cells are harvested by acid precipitation followed by depth filtration. The temperature during depth filtration is performed can be about 20±3° C., 19±3° C., 18±3° C., 17±3° C., 16±3° C., 15±3° C., 14±3° C., 13±3° C., 12±3° C., 11±3° C., 10±3° C., 9±3° C., 8±3° C., 7±3° C., 6±3° C., 5±3° C., or 4±3° C. In one embodiment, depth filtration is followed by pH neutralization. Neutralization can be performed using any base, such as Tris base. Neutralization can be performed at a temperature of about 20±3° C., 19±3° C., 18±3° C., 17±3° C., 16±3° C., 15±3° C., 14±3° C., 13±3° C., 12±3° C., 11±3° C., 10±3° C., 9±3° C., 8±3° C., 7±3° C., 6±3° C., 5±3° C., or 4±3° C. The target pH for neutralization can be approximately 8.0, approximately 7.5, approximately 7.0, approximately 6.5, or approximately 6.0. In one embodiment, the pH is approximately 7.0, approximately 7.1, approximately 7.2, approximately 7.3, approximately 7.4, approximately 7.5, approximately 7.6, approximately 7.7, approximately 7.8, or approximately 7.9. In one embodiment, the pH is 6.0-8.0, 6.5-8.0, or 7.0 to 8.0. In another embodiment, the pH is 8.0±0.2, 7.9±0.2, 7.8±0.2, 7.7±0.2, 7.6±0.2, 7.5±0.2, 7.4±0.2, 7.3±0.2, 7.2±0.2, 7.1±0.2, 7.0±0.2, 6.9±0.2, 6.8±0.2, 6.7±0.2, 6.6±0.2, 6.5±0.2, 6.4±0.2, 6.3±0.2, 6.2±0.2, 6.1±0.2, or 6.0±0.2.

In another embodiment, depth filtration is followed by pH neutralization and viral inactivation, such as viral inactivation with the use a detergent (e.g., Triton). Viral inactivation may be performed at 20±3° C., 19±3° C., 18±3° C., 17±3° C., 16±3° C., 15±3° C., 14±3° C., 13±3° C., 12±3° C., 11±3° C., 10±3° C., 9±3° C., 8±3° C., 7±3° C., 6±3° C., 5±3° C., or 4±3° C. In some embodiments, viral inactivation is not performed after depth filtration.

Accordingly, in one embodiment, the host cells are cultured by fed-batch or perfusion, and then harvested by acid precipitation followed by depth filtration and pH neutralization. In another embodiment, the host cells are cultured by fed-batch or perfusion, and then harvested by acid precipitation followed by depth filtration, pH neutralization, and viral inactivation.

After harvesting, aflibercept may be purified from the cell lysate with Protein A affinity chromatography, followed by anion exchange chromatography (AEX) and then cation exchange chromatography (CEX). In another embodiment, purification is with Protein A affinity chromatography, followed by anion exchange chromatography (AEX) and then hydrophobic interaction chromatography (HIC). In some embodiments, the method comprising AEX followed by HIC produces a lower amount of Y92L clipped species as compared to the same method but with CEX instead of HIC. In some embodiments, the method comprising AEX followed by HIC produces more than two-fold less Y92L clipped species than the same method but with CEX instead of HIC. In some embodiments, the method comprising AEX followed by HIC does not include a CEX step. In some embodiments, the method does not comprise any size exclusion chromatography (SEC) steps. In some embodiments, the method does not comprise any CEX step before AEX.

In some embodiments, a viral inactivation and/or filtration step is performed between the Protein A and AEX steps. In some embodiments, a viral inactivation and/or viral filtration step is performed after the final column purification (e.g., CEX or HIC). In one embodiment, viral filtration is performed after the final column purification (e.g., CEX or HIC).

In some embodiments, the viral inactivation, such as after Protein A purification, is with low pH. In one embodiment, viral inactivation is at a pH of approximately 4.0, approximately 3.9, approximately 3.8, approximately 3.7, approximately 3.6, approximately 3.5, approximately 3.4, approximately 3.3, approximately 3.2, approximately 3.1, or approximately 3.0. In one embodiment, acid precipitation is at a pH of 3.0-4.0 or 3.5-4.0. In another embodiment, the pH is 4.0±0.2, 3.9±0.2, 3.8±0.2, 3.7±0.2, 3.6±0.2, 3.5±0.2, 3.4±0.2, 3.3±0.2, 3.2±0.2, 3.1±0.2, or 3.0±0.2. In some embodiments, following low pH viral inactivation is neutralization, in which the target pH for neutralization is approximately 5.5, approximately 5.0, or approximately 4.5. In one embodiment, the pH is 4.5-6.0, 4.5-5.5, 5.0-6.0, or 5.0-5.5. In another embodiment, the pH is 6.0±0.2, 5.9±0.2, 5.8±0.2, 5.7±0.2, 5.6±0.2, 5.5±0.2, 5.4±0.2, 5.3±0.2, 5.2±0.2, 5.1±0.2, 5.0±0.2, 4.9±0.2, 4.8±0.2, 4.7±0.2, 4.6±0.2, or 4.5±0.2. In some embodiments, depth filtration is performed after neutralization.

In some embodiments, ultrafiltration/diafiltration (UF/DF) is performed after chromatography and/or viral inactivation/filtration and/or depth filtration. In some embodiments, an excipient, such as a surfactant (e.g., a polysorbate) is added to the UF/DF recovery pool, and optionally filtered.

The chromatography, viral inactivation, viral filtration, and/or ultrafiltration/diafiltration steps may be performed at 25±5° C., 24±5° C., 23±5° C., 22±5° C., 21±5° C., 20±5° C., 19±5° C., 18±5° C., 17±5° C., 16±5° C., 15±5° C., 14±5° C., 13±5° C., 12±5° C., 11±5° C., or 10±5° C.

Accordingly, in one embodiment, aflibercept can be purified from the harvested cells by Protein A affinity chromatography followed by viral inactivation (e.g., by low pH, followed by neutralization and optionally, depth filtration), AEX, HIC, viral filtration, and UF/DF. In some embodiments, a surfactant (e.g., polysorbate) is added. In some embodiments, purified aflibercept is filtered after a surfactant is added.

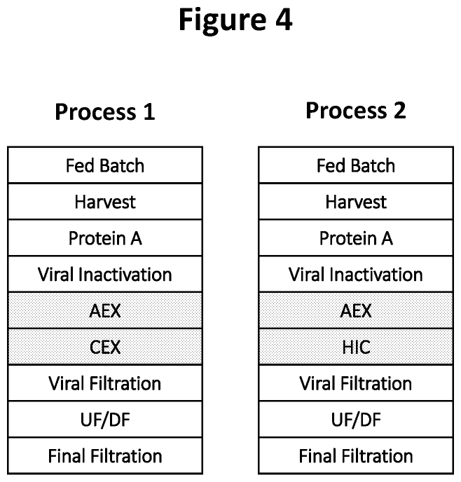

Also provided herein is a method of increasing the binding of a composition comprising a mixture of aflibercept species to PlGF (e.g., PlGF-1 or PlGF-2) and/or VEGF-A comprising reducing the amount of Y92L clipped species in the mixture. In some embodiments, the method comprises purifying aflibercept with an anion exchange chromatography step followed by a hydrophobic interaction chromatography step. In some embodiments, the method is as described herein, such as the second process shown in FIG. 4.

The present disclosure also provides a method of increasing the binding of a composition comprising a mixture of aflibercept species to PlGF (e.g., PlGF-1 or PlGF-2) comprising reducing the N68 occupancy of aflibercept. In some embodiments, the method comprises chromatography steps following Protein A during purification, such as the first or second process as shown in FIG. 4.

The detailed description and following examples illustrate the present invention and are not to be construed as limiting the present invention thereto. Various changes and modifications can be made by those skilled in the art on the basis of the description of the invention, and such changes and modifications are also included in the present invention.

### EXAMPLES

### Example 1: Impact of Attributes of Aflibercept

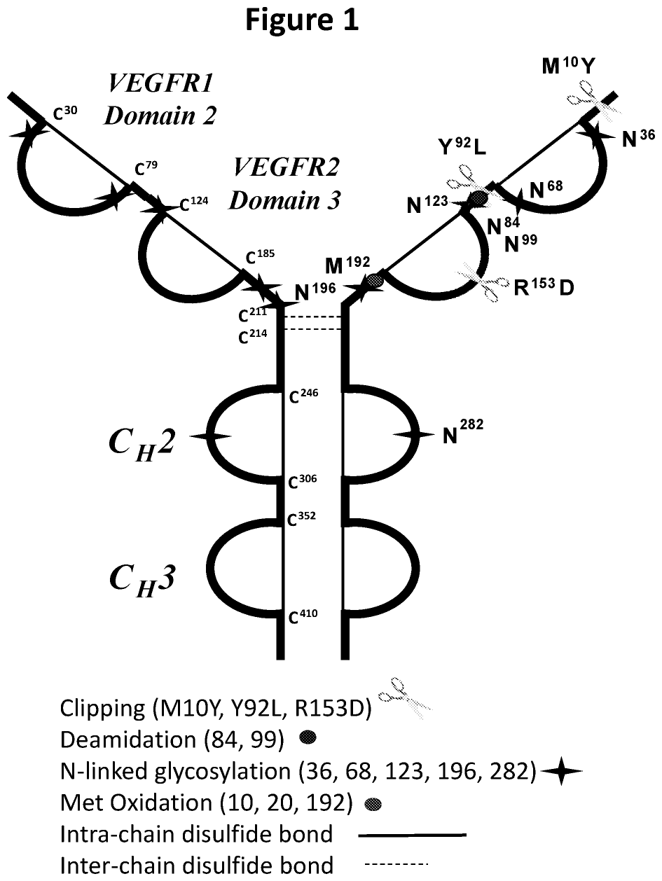

Aflibercept was analyzed for various attributes by the methods shown in Table 1. FIG. 1 depicts these attributes as mapped onto the aflibercept structure. The impact of various attributes on aflibercept's ability to bind VEGF-A and PlGF-1 was determined, in which the results are shown in Table 1.

Peptide mapping characterization or reduced peptide mapping was performed to confirm the amino acid sequences of aflibercept, in addition to assessing the potential presence of chemical and post-translational modifications. Peptide mapping analysis was conducted by enzymatic digestion with trypsin, following reduction with dithiothreitol and alkylation with sodium iodoacetate. The resulting cleavage fragments were separated by reversed phase ultra high-performance liquid chromatography (UHPLC) using an increasing gradient of acetonitrile in water, and the peptides were identified by on-line liquid chromatography mass spectrometry (LC-MS/MS) using a high resolution linear ion trap mass spectrometer.

Hydrophilic interaction liquid chromatography (HILIC) glycan mapping was used to evaluate the N-linked glycans of aflibercept. Aflibercept has five N-linked glycosylation sites, one on the conserved Fc region that is mainly a biantennary complex type, and the other four sites on the VEGF receptor region that are mainly biantennary complex type with sialylated glycan species. The N-linked glycans of aflibercept were evaluated by hydrophilic interaction liquid chromatography (HILIC) UHPLC glycan map analysis. This procedure involves reduction and denaturation of the aflibercept, release of the N-linked glycans with peptide N-glycosidase F (PNGase F), derivatization with a fluorescent label, and fluorescence detection of the labeled glycans separated by HILIC UHPLC using a gradient of increasing ammonium formate in water.

Total sialic acid method was performed to determine the sialic acid content. Terminal sialic acids from the N-linked glycans of aflibercept are hydrolyzed under acidic conditions. The sialic acid in the hydrolyzed solutions are labeled with 1, 2-diamino-4, 5-methyleneoxybenzene (DMB). The preparations of released and labeled sialic acid are diluted with water and subsequently analyzed by ultra high-performance liquid chromatography (UHPLC) with fluorescence detection. A series of injections of known amount of an NANA standard prepared the same way and the corresponding peak areas are used to generate a standard curve using linear regression analysis. The generated linear standard curve is subsequently used to determine the sialic acid content.

Capillary isoelectric focusing (cIEF) analysis of aflibercept was performed using a high resolution capillary electrophoresis separation instrument equipped with a neutral-coated capillary. The electrophoresis of aflibercept through a pH gradient in a capillary allows the aflibercept to migrate until it reaches a pH value equal to its pI. The aflibercept is then chemically mobilized and detected by UV absorbance (280 nm) as it passes through a detection window in the capillary.

Reduced capillary electrophoresis-sodium dodecyl sulfate rCE-SDS was used to evaluate the purity of aflibercept. Samples were reduced using β-mercaptoethanol and denatured with SDS. The reduced and denatured proteins were separated based on hydrodynamic size where smaller size proteins migrate faster, and larger size proteins migrate slower. The analytes were monitored by UV absorbance.

PlGF-1 or PlGF-2 binding was determined using a bead-based Amplified Luminescent Proximity Homogeneous Assay Screen (AlphaScreen®, PerkinElmer) that detects biomolecular interactions. The assay contains two bead types: acceptor beads and donor beads. The donor beads are coated with a hydrogel that contains phthalocyanine, a photosensitizer and streptavidin. The acceptor beads are coated with a hydrogel that contains thioxene derivatives and mouse monoclonal anti-FITC antibody. The donor beads bind to biotinylated PlGF through interaction between streptavidin and biotin, while the acceptor beads bind to FITC-tagged aflibercept. This FITC-tagged aflibercept serves as a competitor for the aflibercept test samples. When FITC-tagged aflibercept and biotinylated PlGF bind to each other, the acceptor beads and the donor beads are brought into close proximity. When a laser is applied to this complex, ambient oxygen is converted to singlet oxygen by the donor beads. If the beads are in close proximity, an energy transfer to the acceptor beads occurs, resulting in the production of luminescence which is measured in a microplate reader equipped with AlphaScreen® signal detection capabilities. When the unconjugated aflibercept test sample is present at sufficient concentrations to inhibit the binding of FITC-tagged aflibercept to the biotinylated PlGF, a dose-dependent decrease in luminescence output occurs. Test sample activity is determined by comparing the test sample response to that of a Reference Standard, representative of relative potency or relative binding (e.g., % relative binding).

VEGFA binding was determined using a bead-based Amplified Luminescent Proximity Homogeneous Assay Screen (AlphaScreen®, PerkinElmer) that detects biomolecular interactions. The assay contains two bead types: acceptor beads and donor beads. The donor beads are coated with a hydrogel that contains phthalocyanine, a photosensitizer and streptavidin. The acceptor beads are coated with a hydrogel that contains thioxene derivatives as well as nickel chelate. The donor beads bind to biotinylated VEGFA-165 through interaction between streptavidin and biotin, while the acceptor beads bind to histidine tagged VEGFR2 due to the interaction between nickel chelate and histidine. When VEGFR2-His and biotinylated VEGFA-165 bind to each other, the acceptor beads and the donor beads are brought into close proximity. When a laser is applied to this complex, ambient oxygen is converted to singlet oxygen by the donor beads. If the beads are in close proximity, an energy transfer to the acceptor beads occurs, resulting in the production of luminescence which is measured in a microplate reader equipped with AlphaScreen® signal detection capabilities. Aflibercept binds to biotinylated VEGFA-165 and prevents it from binding to VEGFR2-His, thereby decreasing the luminescence output in a dose-dependent manner. Test sample activity is determined by comparing the test sample response to that of a Reference Standard, representative of relative potency or relative binding (e.g., % relative binding).

TABLE 1

| | | VEGFA | P1GF1 |
|---|---|---|---|
| Attribute | Methods | Binding* | Binding* |
| N84/N99 Deamidation | LC-MS based peptide | No Impact | No Impact |
| (high pH stress up | mapping | | |
| to 40%) | | | |
| Sialic Acid (Sialidase | LC-MS based peptide | No Impact | Increased |
| digest to remove all | mapping/HPLC/HILIC | | |
| sialic acid) | glycan map | | |
| Y92L Clip (between | LC-MS based peptide | Decreased | Decreased |
| VEGFR1 and VEGR2) | mapping/rCE-SDS | | |
| R153D Clip (on the | rCE-SDS | No Impact | Not |
| VEGR2 disulfide loop) | | | conclusive |
| N68 Glycosylation | LC-MS based peptide | No Impact | Decreased |
| (Occupancy) | mapping/rCE-SDS | | |
| N36 Deglycosylated | LC-MS based peptide | No Impact | No Impact |
| (PGNaseF digest) | mapping | | |
| Fc region | LC-MS based peptide | No Impact | No Impact |
| (FabRICATOR digest) | mapping | | |
| Met Oxidation (TBHP | LC-MS based peptide | No Impact | Not |
| stress up to 40%) | mapping | | determined |

*With increased level of the attribute

As shown in Table 1, other than sialylation, the attributes that affected aflibercept's ability to bind VEGF-A and PlGF-1 are Y92L clipping and N68 glycosylation occupancy.

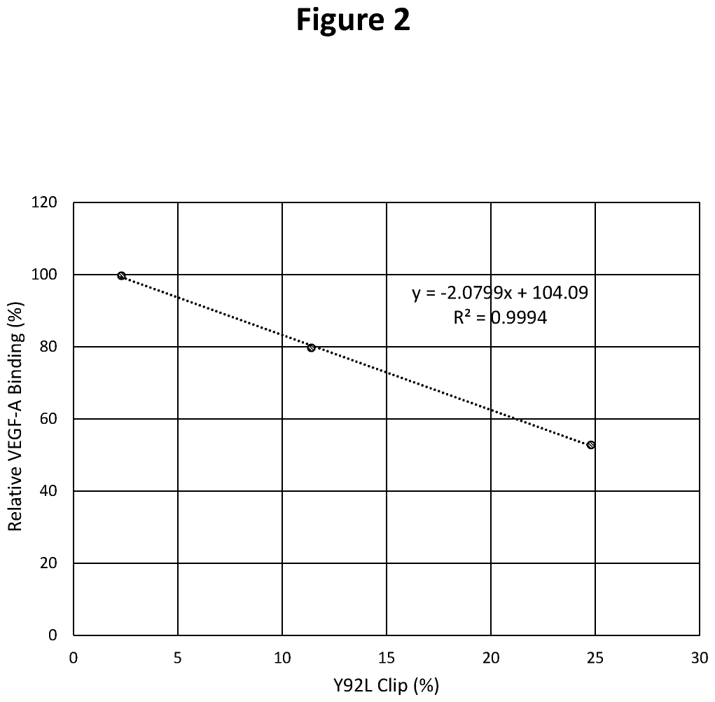

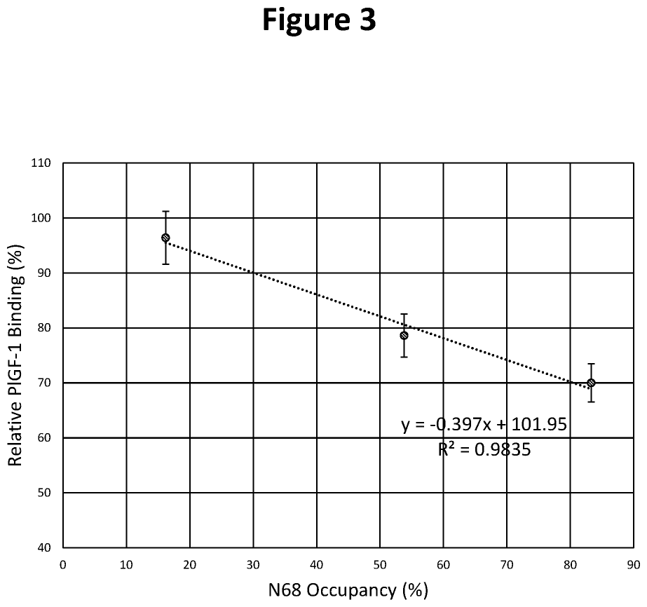

As shown in FIG. 2, Y92L clipping significantly impacts the ability of aflibercept to bind VEGF-A. Also, 1% of Y92L clipping results in 2% VEGF-A binding loss, indicating both VEGFR regions of aflibercept have a role in VEGF-A binding. As shown in FIG. 3, a 10% increase of N68 occupancy results in a 4-5% decrease in the PlGF-1 binding activity of aflibercept.

### Example 2: Process Steps Impacting Y92L Clipping and N68 Occupancy of Aflibercept

As increased Y92L clipping decreases the VEGF-A, PlGF-1 and PlGF-2 binding activity of aflibercept and increased N68 occupancy decreases PlGF-1 and PlGF-2 binding activity of aflibercept, the purification process for aflibercept was investigated to determine whether certain steps in the process could affect the level of Y92L clipped species and the amount of N68 occupancy of aflibercept.

Aflibercept was expressed in Chinese Hamster Ovary (CHO) cells and produced by fed-batch. The cells were then harvested by acid precipitation followed by depth filtration. The pH was then neutralized before a viral inactivation step. Next, aflibercept was purified from the harvested cell culture fluid with Protein A column and then subjected to a viral inactivation step.

After this second viral inactivation step, one process used anion exchange chromatography (AEX) followed by cation exchange chromatography (CEX), while a second process used AEX followed by hydrophobic interaction chromatography (HIC) (FIG. 4). In both processes, these chromatography steps were followed by viral filtration and ultrafiltration/diafiltration (UF/DF) of the viral filtration pool. Polysorbate is then added to the UF/DF recovery pool and then filtered into a container.

The fed-batch process yielded ˜60% of aflibercept that met specific quality attributes. The first purification process (AEX followed by CEX) had higher yield than the second process (AEX followed by HIC). Table 2 shows the percentage of Y92L clip species, percentage of aflibercept with N68 occupancy, the total sialic acid concentration (mol/mol protein), and percentage of cIEF peak 1 (which refers to a group of cIEF peaks which are the low sialic acid-containing versions of aflibercept) of aflibercept prior to AEX, after the first process, and after the second process. Also included in Table 2 is the percent yield of aflibercept across the column 2 and column 3 chromatography steps (AEX and CEX for the first process and AEX and HIC for the second process).

TABLE 2

| | Y92L | N68 | Total SA | cIEF | | % Yield for |
|---|---|---|---|---|---|---|
| | Clip | Occupancy | (mol/mol | peak 1 | % Yield for | Column 3 |
| Process | (%) | (%) | protein) | (%) | AEX | (CEX or HIC) |
| Prior to AEX | 4.7 | 39 | 6.8 | 35 | N/A | N/A |
| First Process | 3.0 | 53 | 8.5 | 7 | ~70 | ~80 |
| (AEX →CEX) | | | | | | |
| Second Process | 1.1 | 54-55 | 9.5 | 3 | ~55 | ~50 |
| (AEX →HIC) | | | | | | |

As Table 2 shows, the AEX step increases the N68 occupancy of aflibercept and the HIC step significantly reduces the Y92L clipped species of aflibercept. AEX can reduce cIEF peak 1 and increase N68 occupancy, whereas HIC can reduce Y92L clip and increase N68 occupancy.

### Example 3: rCE-SDS Analysis of Aflibercept

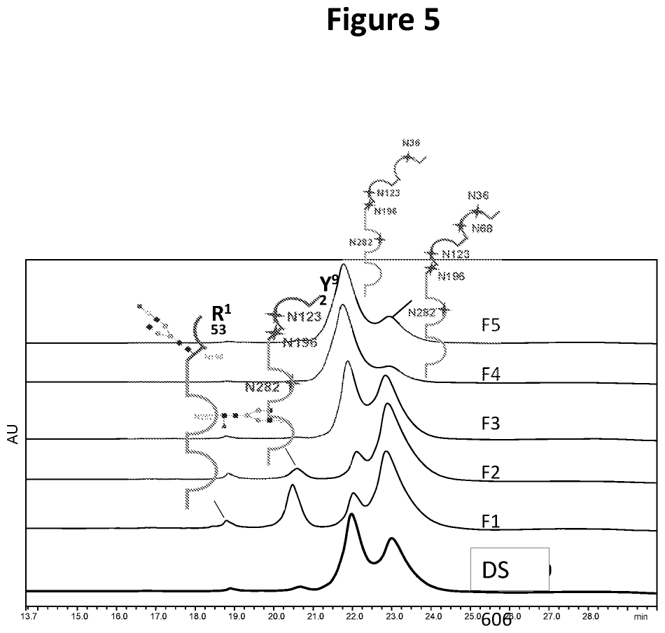

A sample of aflibercept (DS) manufactured with a process using anion exchange chromatography followed by cation exchange chromatography (CEX) was subjected to HIC analytical HPLC and five fractions were collected across the HIC elution peak. These fractions were analyzed by reduced capillary electrophoresis-sodium dodecyl sulfate (rCE-SDS) (FIG. 5), which shows that HIC HPLC partially resolves clipped aflibercept species and N68 glycosylated species.

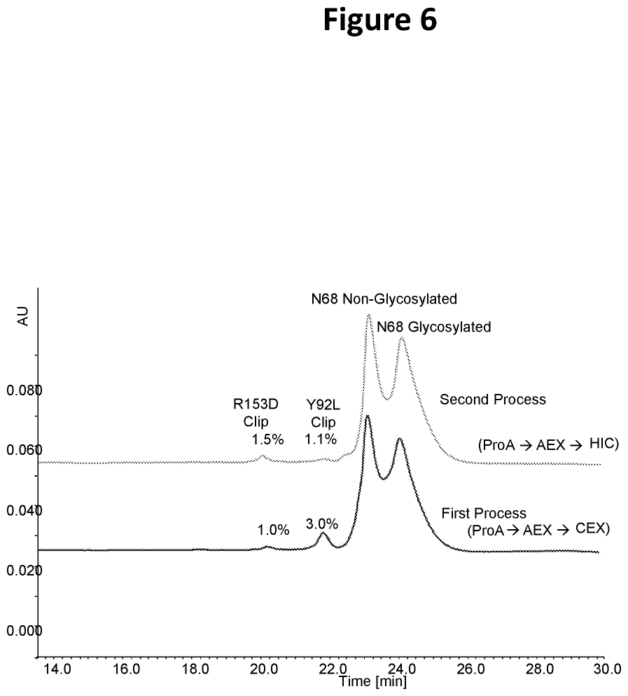

rCE-SDS of the aflibercept produced by the first process as compared to aflibercept produced by the second process is shown in FIG. 6, showing that the yield of Y92L clipped species is lower using the second process as compared to the first process, while the second process had higher N68 occupancy as compared to the first process.

### Example 4: Impact of Attributes on Aflibercept Binding VEGF-A, PlGF-1, and PlGF-2

The percentage of aflibercept purified from a process using Protein A and column and cation exchange chromatography (CEX) that was cleaved at Y92L, and the percentage of aflibercept purified from a process using Protein A and column and cation exchange chromatography (CEX) in which N68 is glycosylated (N68 occupancy) was determined for each of the analytical HPLC HIC fractions shown in FIG. 5, using rCE-SDS. Each fraction was also subjected to VEGF-A and PlGF-1 and PlGF-2 binding assays. The results are shown in Table 3.

TABLE 3

| | Y92L | N68 | Relative | Relative | Relative |
|---|---|---|---|---|---|
| Frac- | Clip | Occupancy | VEGFA | P1GF-1 | PIGF-2 |
| tions | (%) | (%) | Binding (%) | Binding (%) | Binding (%) |
| HIC F1 | 20.5 | 79.7 | 53 | 44 | 66 |
| HIC F2 | 7.5 | 83.3 | 80 | 55 | 81 |
| HIC F3 | 0.8 | 53.8 | 100 | 77 | 86 |
| HIC F4 | 0.7 | 16.2 | 100 | 96 | 150 |

As shown in Table 3, clipping of aflibercept at Y92L impacts VEGF-A and PlGF binding. Fractions with a higher percentage of Y92L clipped species showed lower binding to VEGF-A, PlGF-1 and PlGF-2. Meanwhile, as shown in Table 3, N68 occupancy impacts PlGF binding, but not VEGF-A binding. Fractions with a higher percentage of species in which N68 is glycosylated showed lower binding to PlGF-1 and PlGF-2.

### Example 5: Glycan Profile of Aflibercept

The glycan profile of aflibercept purified from a process using Protein A followed by cation exchange chromatography (CEX) was determined by rCE-SDS (% N-glycosylation at N68) and LC-MS based peptide mapping (e.g., the other attributes listed in Table 4).

TABLE 4

| Domain | Attribute | Aflibercept |
|---|---|---|
| VEGFR domains glycan: | Sialic acid at N⁶⁸ | 0.7 |
| sialylation | (mol/mol protein)^{a} | |
| VEGFR domains glycan: | % O-glycosylation at T³³ | 4 |
| occupancy | % N-glycosylation at N³⁶ | 95 |
| | % N-glycosylation at N⁶⁸ | 26 |
| Fc domain glycan groups | % high mannose | 1 |
| | % afucosylation | 6 |
| | % galactosylation | 26 |
| | % sialylation | 1 |

^{a}The average number of sialic acid per protein is calculated from the average number of sialic acid per polypeptide by multiplying by a factor of 2 (since the peptide map procedure results in the reduction of aflibercept producing 2 polypeptide chains).

The glycan profile of aflibercept purified from a process using Protein A followed by anion exchange chromatography (AEX) and hydrophobic interaction chromatography (HIC) was determined by rCE-SDS (% N-glycosylation at N68 and Y92L clipped species) and LC-MS based peptide mapping (e.g., the other attributes listed in Table 5).

TABLE 5

| Domain | Analytical Testing/Attribute | Aflibercept |
|---|---|---|
| VEGFR domains glycan: | Sialic acid at N⁶⁸ | 0.8 |
| sialylation | (mol/mol protein)^{a} | |
| VEGFR domains glycan: | % O-glycosylation at T³³ | 8.3 |
| occupancy | % N-glycosylation at N³⁶ | 90.9 |
| | % N-glycosylation at N⁶⁸ | 51.9 |
| Fc domain glycan groups | % high mannose | 0.4 |
| | % afucosylation | 4.0 |
| | % galactosylation | 24.4 |
| | % sialylation | 3.8 |
| VEGFR domains | % Y92L clip | 0.4 |

^{a}The average number of sialic acid per protein is calculated from the average number of sialic acid per polypeptide by multiplying by a factor of 2 (since the peptide map procedure results in the reduction of ABP 938 producing 2 polypeptide chains).

### Example 6: Impact of Temperature and pH Shifts on Y92L Clipping

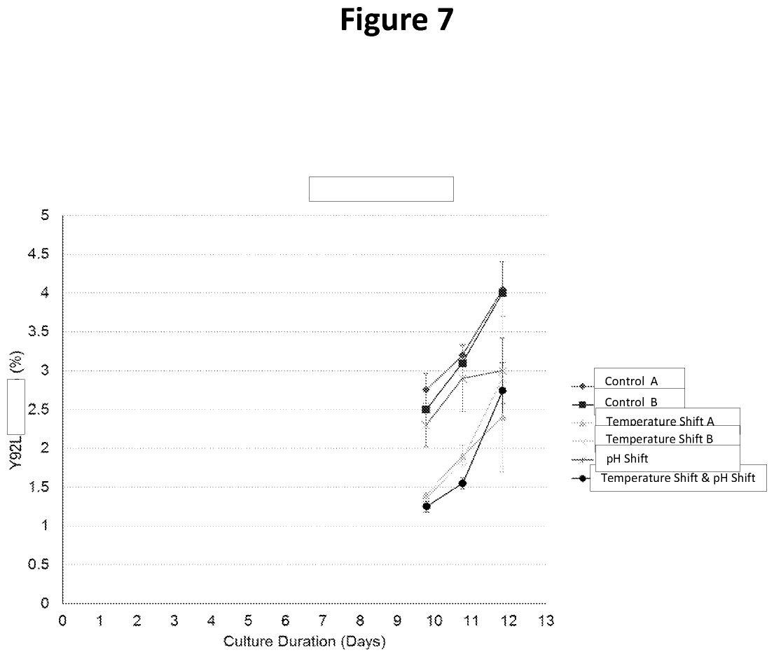

Fed-batch bioreactor cultures of CHO cells expressing aflibercept were subjected to shifts in temperature and/or pH on Day 6 of culture and compared to cultures that were not shifted. Two control cultures (FIG. 7, Control A, Control B), each in a different bioreactor, were grown at 36° C., pH 6.9 until harvest. Two cultures (FIG. 7, Temperature Shift A, Temperature Shift B), each in a different bioreactor, were grown at 36° C., pH 6.9 and on Day 6 of culturing, the temperature for both was lowered to 32.5° C. and maintained at 32.5° C., pH 6.9 until harvest. One culture (FIG. 7, pH Shift) was grown at 36° C., pH 6.9 and the pH was lowered to 6.8 on Day 6 of culturing, and maintained at 36° C., pH 6.8 until harvest. One culture (FIG. 7, Temperature Shift & pH Shift) was grown at 36° C., pH 6.9 and both the temperature and pH was lowered to 32.5° C., pH 6.8 on Day 6 of culturing, and maintained at 32.5° C., pH 6.8 until harvest.

Aflibercept was purified from the clarified culture broths of each culture using Protein A, and the percentage of Y92L clipped species determined by rCE-SDS on Days 10, 11 and 12 (FIG. 7). For the all of cultures, the percentage of Y92L clipped species increased with culture time. Shifting the culture temperature and/or pH to lower values at Day 6 lowered the percentage of Y92L clipped species compared to the controls (Control A, Control B). Temperature shifting was more effective in lowering the percentage of Y92L clipped species than pH shifting and there was no increased effect in combining pH and temperature shifts to further lower the percentage of Y92L clipped species.

While the present invention has been described in terms of various embodiments, it is understood that variations and modifications will occur to those skilled in the art. Therefore, it is intended that the appended claims cover all such equivalent variations that come within the scope of the invention as claimed. In addition, the section headings used herein are for organizational purposes only and are not to be construed as limiting the subject matter described.

All references cited in this application are expressly incorporated by reference herein for any purpose.

1. A Y92L clipped species of aflibercept comprising SEQ ID NO: 3.

2. A composition comprising a mixture of aflibercept species, wherein the amount of Y92L clipped species in the composition is less than 5.0%, as determined by rCE-SDS and/or at least 30% of the aflibercept species is occupied at position N68, as determined by rCE-SDS.

3. The composition of claim 2, wherein the amount of Y92L clipped species in the composition is less than 0.8%, or about 0.4%.

4.-5. (canceled)

6. The composition of claim 2, wherein at least 50%, between 50-60%, or about 39%, 53%, 54%, or 55% of the aflibercept species is occupied at position N68.

7.-8. (canceled)

9. The composition of claim 2, wherein the amount of Y92L clipped species in the mixture is between 1% and 3%, less than 0.8%, or about 0.4%. (claims 10-11)

10.-11. (canceled)

12. The composition of claim 2, wherein between 54-55% of the aflibercept species is occupied at position N68 and wherein the amount of Y92L clipped species in the mixture is about 1.1%, or between 54-55% of the aflibercept species is occupied at position N68 and wherein the amount of Y92L clipped species in the mixture is about 0.4%.

13. (canceled)

14. A composition comprising a mixture of aflibercept species, wherein the total sialic acid content of aflibercept species is between 6.0 and 10.0 mol/mol protein, as determined by LC-MS based peptide mapping method and/or the aflibercept species comprise between 1.0-12% afucosylation in the Fc domain.

15. The composition of claim 14, wherein the total sialic acid content is about 6.8, 8.5 or 9.5 (mol/mol protein).

16.-18. (canceled)

19. The composition of claim 14, wherein the aflibercept species comprise less than 10%, about 6%, less than 5%, or about 4% afucosylation in the Fc domain.

20.-22. (canceled)

23. A composition comprising a mixture of aflibercept species, wherein the aflibercept species comprise: [claims 24-26]
(I)
a) between 0.1-2.0 (mol/mol protein) of sialic acid at N68;
b) between 1.0-25.0% O-glycosylation at T33;
c) between 70%-99% N-glycosylation at N36;
d) between 20%-75% of N-glycosylation at N68;
e) between 0.1-1.0% high mannose in the Fc domain;
f) between 26% galactosylation in the Fc domain;
g) about 1% sialylation in the Fc domain; or
h) any combination thereof
(II)
a) about 0.7 (mol/mol protein) of sialic acid at N68;
b) about 4% O-glycosylation at T33;
c) about 95% N-glycosylation at N36;
d) about 26% of N-glycosylation at N68;
e) about 1% high mannose in the Fc domain;
f) about 26% galactosylation in the Fc domain;
g) about 1% sialylation in the Fc domain; or
i) any combination thereof; or
(III)
a) about 0.8 (mol/mol protein) of sialic acid at N68;
b) about 8.3% O-glycosylation at T33;
c) about 90.9% N-glycosylation at N36;
d) about 51.9% of N-glycosylation at N68;
e) about 0.4% high mannose in the Fc domain;
f) about 24.4% galactosylation in the Fc domain;
g) about 3.8% sialylation in the Fc domain; or
j) any combination thereof.

24.-25. (canceled)

26. The composition of claim 23, wherein the amount of Y92L clipped species in the mixture is about 0.4%.

27. A method of increasing the binding of a composition comprising a mixture of aflibercept species to PlGF and/or VEGF-A comprising reducing the amount of Y92L clipped species in the mixture or the N68 occupancy of aflibercept.

28.-30. (canceled)

31. The method of claim 27, wherein the method comprises purifying aflibercept with an anion exchange chromatography step followed by a hydrophobic interaction chromatography step.

32. The method of claim 35,
wherein a lower amount of Y92L clipped species is produced as compared to a purification process comprising an AEX step followed by a cation exchange chromatography (CEX) step.

33. (canceled)

34. A method of producing a composition comprising a mixture of aflibercept species comprising:
a) harvesting cells producing aflibercept;
b) subjecting the harvested cell culture fluid to a Protein A column; and
c) performing anion exchange chromatography (AEX) followed by cation exchange chromatography (CEX) or hydrophobic interaction chromatography (HIC).

35. The method of claim 34, wherein performing AEX is followed by performing HIC, and the method does not include performing CEX.

36.-37. (canceled)

38. The method of claim 35, wherein the cells are Chinese Hamster Ovary (CHO) cells.

39. The method of claim 35, wherein the cells are grown by perfusion or fed-batch.

40. The method of claim 39, wherein the cells are harvested by acid precipitation, centrifugation, depth filtration or any combination thereof.

41. The method of claim 40, further comprising one or more viral inactivation and/or viral filtration step(s).

42. The method of claim 41, further comprising a ultrafiltration/diafiltration (UF/DF) step.

43.-44. (canceled)
